(19) United States
(12) Patent Application Publication
(10) Pub. No.: US 2023/0285347 A1
(43) Pub. Date: Sep. 14, 2023

(54) Preterm Labour with Prostaglandin E2 Receptor Agonists

(71) Applicant: IMPERIAL COLLEGE INNOVATIONS LIMITED, London (GB)

(72) Inventors: Abigail Walker, London (GB); Phillip Bennett, London (GB); Aylin Hanyaloglu, London (GB)

(21) Appl. No.: 18/007,417

(22) PCT Filed: Jul. 30, 2021
(86) PCT No.: PCT/GB2021/051971

§ 371 Date: Jan. 30, 2023

(30) Foreign Application Priority Data
Jul. 31, 2020 (GB) 2011941.8

(51) Int. Cl. A61K 31/216; A61P 15/06

(52) U.S. Cl. CPC A61K 31/216; A61P 15/06

(57) ABSTRACT

The present invention relates to compounds for use in the delaying of labour and/or inhibition of uterine contractions in pre-term pregnant subjects. Such compounds include selective prostaglandin EP2 receptor agonists which specifically activate the anti-labour cyclic AMP (cAMP) pathways of EP2, without inducing signals that induce pro-labour G protein-dependent pro-inflammatory pathways, as observed with other conventional EP2 agonists within the art. The invention also refers to methods and uses of such compounds.

### FIELD OF INVENTION

The following invention relates to compounds for use in the management of preterm labour.

### BACKGROUND

Preterm labour leading to preterm birth is the number one killer globally for children under the age of 5. Infants born at less than 28 weeks spend 85 times longer in hospital as full-term babies during the first 5 years of life, with substantial healthcare costs. Despite advances in neonatal care and tocolytic drugs, the frequency of preterm birth in most Western countries is increasing.

Of 600,000 UK live births per annum, approximately 8,000 will be at birth weights below 1500 g, which equates to a gestational age of less than 32 weeks. Of these preterm babies, 1600 will die and a further 600 will develop cerebral palsy. The impact of disability increases dramatically when delivery occurs close to the limits of viability at around 24 weeks. The EPICURE study described how 25% of babies born before 25 weeks who survive to be discharged from hospital develop severe disability, 25% mild disability and less than 50% are developmentally normal at 30 months of age (Wood et al. 2000, The New England Journal of Medicine 343: 378-384). The economic impact of providing long-term health and social care for these families is therefore significant.

Preterm birth is defined as childbirth occurring at less than 37 completed weeks or 259 days of gestation. Children born prematurely have higher rates of cerebral palsy, sensory deficits, learning disabilities and respiratory illness. About 28% of neonatal deaths are caused due to PTL. In the lower-income countries, on average, 12% of babies are born too early compared with 9% in higher-income countries (http://www.who.int/mediacentre/factsheets/fs363/en/). Over 60% of preterm births occurred in sub-Saharan Africa and South Asia where 9.1 million births (12.8%) annually are estimated to be preterm. The rates are highest for low-income countries (11.8%), followed by lower middle-income countries (9.4% and 9.3%). Globally more than 1 in 10 births will be preterm. Over the past 20-30 years the incidence of preterm birth in most developed countries has been about 5-7% of live births. The incidence is about 12% in the U.S. and in the UK 60,000 are born prematurely annually (BMJ 2004; 329:675; tommys.org).

Tocolytic drugs, i.e. drugs that inhibit contractions of women in labour, are limited to agonists of the beta-adrenergic receptors and antagonists of the oxytocin receptor, namely atosiban. These are designed to only temporarily inhibit contractions (max 48 hours) to enable transport of the patient to a specialist neonatal ICU and administration of steroids to develop the immature infant lung before delivery. However, use of beta-adrenergic receptor agonists has decreased due to adverse effects on the mother particularly on the cardio-vascular system. Atosiban has a small but significant increase over placebo in inhibiting contractions and is deemed safer than beta-adrenergic receptor mimetics. However, this oxytocin receptor antagonist was then discovered to be a biased agonist that still activates G protein-dependent pro-inflammatory pathways, and potentially exposing the uterus to a prolonged inflammatory environment that could be adverse to the developing lung and brain. In addition, this peptide antagonist/biased agonist is expensive to produce.

Similarly, agonists for the prostaglandin EP2 receptor (EP2) has been used as a form of temporary tocolytic control at full-term labour. Butaprost activates EP2, which signals via both anti-labour and pro-labour (pro-inflammatory) pathways, of which only the pro-labour pathway is maintained following the onset of labour (Kandola et al. 2014, Endocrinology). Thus, the problem exists that butaprost, as a secondary effect, also targets the pro-inflammatory signalling pathway in the myometrium, contributing towards pro-labour mechanisms.

Therefore, there are no current tocolytics that offer safe, efficacious and cost-effective treatment, and none so far have been able to prolong pregnancy beyond a few days and also target pro-inflammatory environment of the in-labour preterm uterus.

### SUMMARY OF INVENTION

This invention is based on the surprising discovery that preterm labour in a subject can be managed through the use of selective prostaglandin EP2 receptor (EP2) agonists, wherein such agonists only activate the anti-labour cyclic AMP (cAMP) pathways of EP2, without inducing signals that induce pro-labour G protein-dependent pro-inflammatory pathways. As such, the selective agonists may be used as a safe, efficacious, cost-effective and long-acting treatment for preterm labour.

In a first aspect of the invention, there is provided a compound for use in delaying labour and/or inhibiting uterine contractions in a preterm pregnant subject, wherein the compound is a compound of Formula (I):

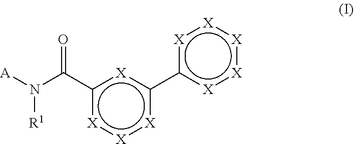

or a pharmaceutically acceptable salt, solvate, hydrate, tautomer, optical isomer, N-oxide, and/or prodrug thereof,
wherein A is:

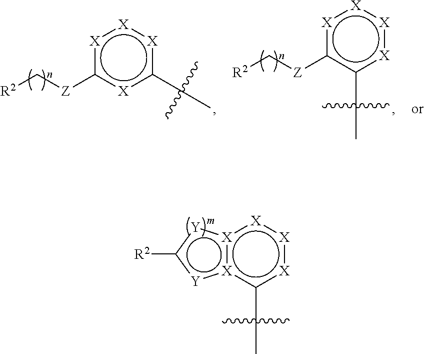

m is 1 or 2;
each X and Y is independently selected from C, N, O, and S, wherein each X and Y is optionally independently substituted with a substituent selected from the group consisting of:
(i) C₁-C₆ alkyl, C₂-C₆ alkenyl, C₂-C₆ alkynyl, C₁-C₆ alkoxy, and (C₁-C₃ alkyl)-O—(C₁-C₃ alkyl), each of which is optionally substituted with one or more substituents independently selected from halo, —OH, —NR³R⁴, and oxo; and
(ii) halo, —OH, —CN, —NO₂, —SO₂, —NR³R⁴, —C(O)NR³R⁴—C(O)OR⁵, and —C(O)R⁵;
each Z is independently selected from the group consisting of —CH₂—, —O—, —S—, —NH—, and —C═C—, preferably —CH₂—, —O—, and —C═C—;
each n is independently 0, 1, 2, or 3;
R¹ is H or is selected from the group consisting of C₁-C₆ alkyl, C₂-C₆ alkenyl, C₂-C₆ alkynyl, each of which is optionally substituted with one or more substituents independently selected from halo, —NR³R⁴, —OH, and oxo;
each R² is independently selected from the group consisting of —C(O)OH, —C(O)OR⁶, —OC(O)R⁶, —C(O)R⁶, —C(O)NHR⁶, —NHC(O)R⁶, —OR⁶, —R⁶, and 5-membered heteroaryl, wherein the 5-membered heteroaryl is optionally substituted with one or more substituents independently selected from C₁-C₆ alkyl and halo, the C₁-C₆ alkyl being optionally substituted with one or more halo atoms;
R³, R⁴, and R⁵ are each independently selected from the group consisting of H, and C₁-C₆ alkyl, wherein the C₁-C₆ alkyl is optionally substituted with one or more halo atoms;
each R⁶ is independently selected from the group consisting of H and C₁-C₆ alkyl, wherein the C₁-C₉ alkyl is optionally substituted with one or more substituents independently selected from halo and —OH.

In a second aspect of the invention, there is provided a selective prostaglandin E2 receptor (EP2) agonist for use in delaying labour and/or inhibiting uterine contractions in a preterm pregnant subject.

In a third aspect of the invention, there is provided a pharmaceutical formulation for use in delaying labour and/or inhibiting uterine contractions in a preterm pregnant subject, wherein the pharmaceutical composition comprises a compound of Formula (I) as defined in the first aspect and one or more pharmaceutically acceptable carriers or excipients.

In a fourth aspect of the invention, there is provided a method for delaying labour and/or inhibiting uterine contractions in a preterm pregnant subject, the method comprising the step of administering to a patient in need thereof a therapeutically effective amount of a compound of Formula (I) as defined in the first aspect, an agonist as defined in the second aspect, or a pharmaceutical composition as defined in the third aspect of the invention.

In a fifth aspect of the invention, there is provided a use of a compound of Formula (I) as defined in the first aspect, an agonist as defined in the second aspect, or a pharmaceutical composition as defined in the third aspect, in the manufacture of a medicament for delaying labour and/or inhibiting uterine contractions in a preterm pregnant subject.

### DESCRIPTION OF FIGURES

FIG. 1 shows the EC50 values for EP2 agonist-mediated induction of cAMP, in HEK 293 cells stably expressing human FLAG-tagged EP2. PGN9856i exhibited the greatest potency, by three orders of magnitude.

FIG. 2 shows the maximal dose for each ligand. All EP2 ligands elicited intracellular cAMP release in HEK 293 cells stably expressing EP2. Data represent mean±SEM from at least 3 independent experiments. One-way ANOVA with Tukey post-hoc comparison. ****p<0.0001 vs DMSO.

FIG. 3 shows the intracellular calcium response of HEK 293 cells following acute butaprost (10 μM), AH13205 (10 μM) or PGN9856i (100 nM) stimulation. Fluorescence intensity normalised to background for each cell is measured (F-FO). N=3. One-way ANOVA with Tukey post-hoc comparison: ***p<0.001. PGN9856i is unable to elicit intracellular calcium release in HEK 293 cells.

FIG. 4 shows the fold change of PGE2, in HEK293 cells stably expressing EP2, stimulate with either butaprost (10 μM), AH13205 (10 μM) or PGN9856i (100 nM) for 6 hours. Tissue culture media collected and used for ELISA. Data shown as fold change over butaprost response, mean±SEM from at least 3 independent experiments. One-sample t-test: ****p<0.0001. One-way ANOVA with Dunnett's post-hoc comparison: ###p<0.001. PGN9856i is unable to elicit PGE2 release in HEK 293 cells.

FIG. 5 shows intracellular cAMP release in primary myometrial cells. HEK 293 cells stably expressing EP2 were treated with IBMX (5 minutes, 0.5 mM) before stimulation with IBMX and either DMSO, butaprost (10 μM), AH13205 (10 μM) or PGN9856i (100 nM) for a further minute. cAMP concentrations were normalised to protein. Data represent mean±SEM from at least 3 independent experiments. One-way ANOVA with Sidak's multiple comparisons test. Basal vs. DMSO, DMSO vs. PGN9856i. All EP2 ligands elicit intracellular cAMP release.

FIG. 6 shows: (A) the intracellular calcium response in primary myometrial cells, following acute stimulation with either butaprost (10 μM), AH13205 (10 μM) or PGN9856i (100 nM). Data shows the mean of the maximum fluorescence intensity for each cell measured normalised to background (F-FO). One-way ANOVA with Dunnet's multiple comparison post-hoc test: **p<0.01, and (B) calcium intensity traces over time from representative cells from (A). PGN9856i is unable to elicit intracellular calcium release in primary myometrial cells.

FIG. 7 shows PGE2 release in primary myometrial cells, stimulated with either DMSO, butaprost (10 μM), AH13205 (10 μM) or PGN9856i (100 nM) for 6 hours. Tissue culture media collected and used for ELISA. Data shown as mean±SEM from at least 3 independent experiments. One-way ANOVA with Dunnet's multiple comparison post-hoc test: *p<0.05, **p<0.01. PGN9856i is unable to elicit PGE2 release in primary myometrial cells.

FIG. 8 shows how EP2-mediated cAMP signalling is reduced with the onset of labour in PO myometrial cells. L=non-labouring, L+E=early stage spontaneous labour, L+L=late stage spontaneous labour, L+SE=early stage induced labour, L+SL=late stage induced labour. Myometrial cells from different labouring groups were treated with IBMX (5 minutes, 0.5 mM) before 1 minute stimulation with IBMX with/without butaprost (10 μM) or isoproterenol (10 μM). cAMP concentrations were normalised to protein. Data shown as mean±SEM, L−n=4. All other groups n=5. Each treatment compared to L− using one-way ANOVA with Dunnett's multiple comparison post-hoc test: **p<0.01, ***p<0.001, ****p<0.0001.

FIG. 9 shows how EP2-mediated inflammatory COX-2 signalling is changed with the onset of labour in PO myometrial cells. L=non-labouring, L+E=early stage spontaneous labour, L+L=late stage spontaneous labour, L+SE=early stage induced labour, L+SL=late stage induced labour. (A) COX-2 protein (70 kB) determined by western blot and normalised to GAPDH protein (38 kB) in different labouring groups following 6 hour butaprost (10 μM) stimulation. Data shown as mean±SEM, n=5. Groups compared using one-way ANOVA with Dunnett's multiple comparison post-hoc test: *p<0.05. (B) Representative western blot for FIG. 9A.

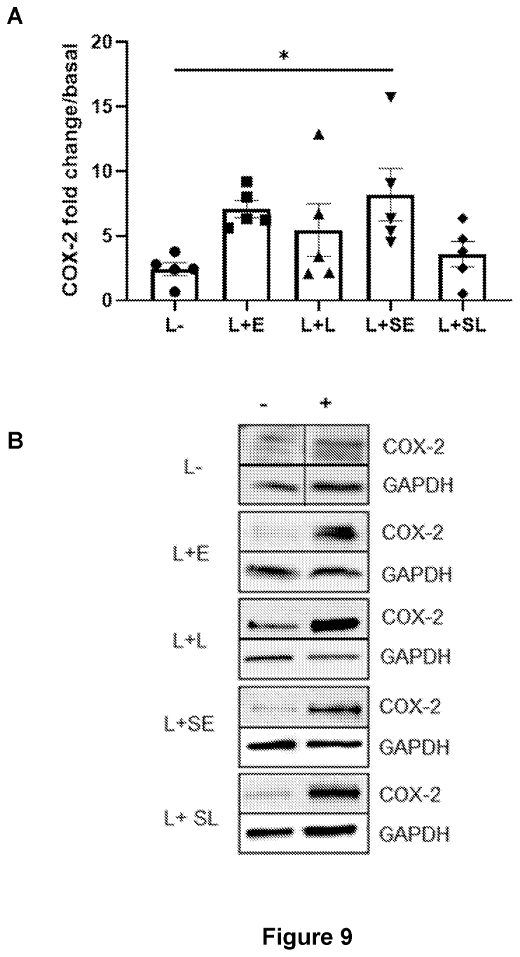

FIG. 10 shows how oxytocin priming of non-labouring myocytes attenuates EP2-mediated cAMP. Myometrial cells with/without oxytocin (OT) pre-treatment (1 hour, 100 nM) were treated with IBMX (5 minutes, 0.5 mM) before 1 minute stimulation with IBMX with/without either DMSO, butaprost (10 μM), AH13205 (10 μM) or PGN9856i (100 nM). cAMP concentrations were normalised to protein. Each patient's data normalised to butaprost response and shown as mean±SEM, n=6. One-sample t-test: #p<0.05. ##p<0.01. One-way ANOVA with Sidak's multiple comparison post-hoc test: **p<0.01.

FIG. 11 shows that oxytocin cannot enhance EP2-mediated calcium when activated with PGN9856i. Intracellular calcium response in myometrial cells with/without OT pre-treatment (1 hour, 100 nM) stimulated with butaprost (10 μM), AH13205 (10 μM) or PGN9856i (100 nM). Data are the mean of the maximum fluorescence intensity for each cell measured normalised to background (F-FO). Shown as fold-change over butaprost response. One-way ANOVA with Dunnett's multiple comparison post-hoc test: ##p<0.01. One-sample t-test: *p<0.05.

FIG. 12 shows that oxytocin cannot enhance EP2-mediated PGE2 when activated with PGN9856i. Myometrial cells stimulated with either DMSO, butaprost (10 μM), AH13205 (10 μM) or PGN9856i (100 nM) for 6 hours, with/without OT pre-treatment (1 hour, 100 nM). Tissue culture media collected and used for ELISA. PGE2 concentrations normalised to butaprost. Data shown as mean±SEM from at least 3 independent experiments. One-way ANOVA with Dunnett's multiple comparison post-hoc test. One-sample t-test: *p<0.05.

FIG. 13 shows that PGN9856i reduces OT-dependent PGE2 release. Myometrial cells stimulated with DMSO or PGN9856i (100 nM) for 6 hours, with/without OT pre-treatment (1 hour, 100 nM). Tissue culture media collected and used for ELISA. PGE2 concentrations normalised to vehicle. Data shown as mean±SEM from at least 3 independent experiments. Unpaired t-test: *p<0.05.

FIG. 14 shows that EP2 signalling is dually coupled to the relaxatory cAMP- and pro-labour COX-2/calcium pathway in preterm and term pregnant myometrium. (A) cAMP levels measured in myocytes from non-labouring term (L−) (39-41 weeks) or preterm L− (27-29 weeks), treated with IBMX (0.5 mM for 5 min) before stimulation with 10 μM butaprost (EP2 agonist) or 10 μM isoproterenol (Iso) for 2 min. Data represent cAMP concentrations as mean+/−SEM. (B) Term L− and Preterm L− cells were treated with calcium sensitive dye, Fluo-4-AM, and after the incubation stimulated with 10 μM butaprost (EP2 agonist) and imaged via confocal microscopy. The maximal fluorescent intensity is expressed as mean+/−SEM. (C) COX-2 levels were measured via western blotting after treatment with 10 μM butaprost (EP2 Ag) for 6 h and normalized to GAPDH and expressed as fold change over basal (mean+/−SEM). For all experiments n=4 in each group.

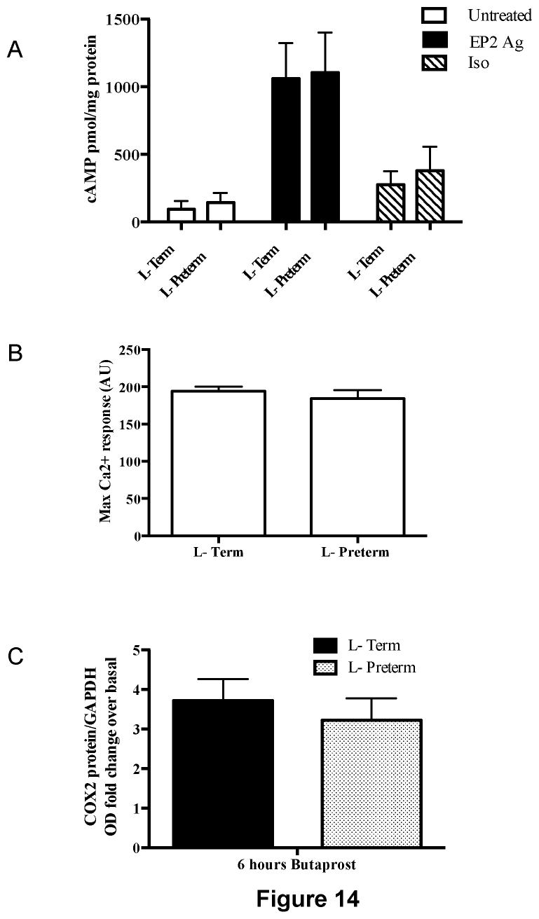

### DETAILED DESCRIPTION OF THE INVENTION

This invention is predicated on the surprising discovery that selective prostaglandin EP2 receptor (EP2) agonists may be used in the treatment and/or management of preterm labour, wherein said selective agonists only activate the anti-labour, myometrium-relaxing, cyclic AMP (cAMP) pathways of the receptor, without inducing signals that increase inflammation, as observed with other conventional EP2 agonists within the art.

Thus, in a first aspect of the invention, there is provided compound for use in delaying labour and/or inhibiting uterine contractions in a preterm pregnant subject, wherein the compound is a compound of Formula (I):

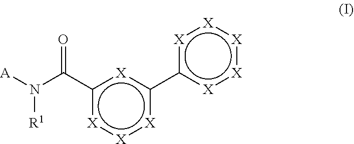

or a pharmaceutically acceptable salt, solvate, hydrate, tautomer, optical isomer, N-oxide, and/or prodrug thereof,
wherein A is:

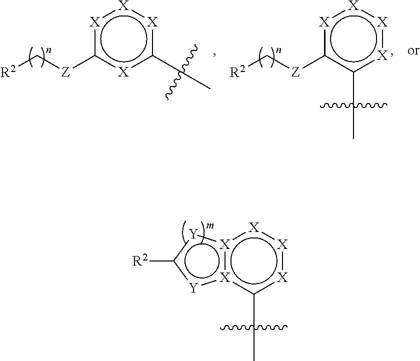

m is 1 or 2;
each X and Y is independently selected from C, N, O, and S, wherein each X and Y is optionally independently substituted with a substituent selected from the group consisting of:
(i) C₁-C₆ alkyl, C₂-C₆ alkenyl, C₂-C₆ alkynyl, C₁-C₆ alkoxy, and (C₁-C₃ alkyl)-O—(C₁-C₃ alkyl), each of which is optionally substituted with one or more substituents independently selected from halo, —OH, —NR³R⁴, and oxo; and
(ii) halo, —OH, —CN, —NO₂, —SO₂, —NR³R⁴, —C(O)NR³R⁴—C(O)OR⁵, and —C(O)R⁵;
each Z is independently selected from the group consisting of —CH₂—, —O—, —S—, —NH—, and —C═C—;
each n is independently 0, 1, 2, or 3;
R¹ is H or is selected from the group consisting of C₁-C₆ alkyl, C₂-C₆ alkenyl, C₂-C₆ alkynyl, each of which is optionally substituted with one or more substituents independently selected from halo, —NR³R⁴, —OH, and oxo;
each R² is independently selected from the group consisting of —C(O)OH, —C(O)OR⁶, —OC(O)R⁶, —C(O)R⁶, —C(O)NHR⁶, —NHC(O)R⁶, —OR⁶, —R⁶, and 5-membered heteroaryl, wherein the 5-membered heteroaryl is optionally substituted with one or more substituents independently selected from C₁-C₆ alkyl and halo, the C₁-C₆ alkyl being optionally substituted with one or more halo atoms;
R³, R⁴, and R⁵ are each independently selected from the group consisting of H, and C₁-C₆ alkyl, wherein the C₁-C₆ alkyl is optionally substituted with one or more halo atoms;
each R⁶ is independently selected from the group consisting of H and C₁-C₆ alkyl, wherein the C₁-C₉ alkyl is optionally substituted with one or more substituents independently selected from halo and —OH.

The term “preterm” as used herein refers to a pregnancy in a subject that is three or more weeks before the full gestation period of about 40 weeks. As such, the term “preterm labour (PTL)” refers to the condition where labour begins three or more weeks before the full gestation period of about 40 weeks (i.e. labour begins at 37 weeks of gestation or less). PTL can lead to a premature (or preterm) birth.

Preterm labour (PTL) is a syndrome, not a single disease process. Some aetiologies, for example placental abruption, are unpredictable and unpreventable. Maternal and/or fetal ‘stress’ may cause preterm labour by cortisol-mediated effects upon placental CRH. Multiple pregnancy causes preterm delivery both through placental CRH and through mechanical stretch of the uterus and cervix. But these are, in general, causes of ‘late’ preterm labour with less severe medical and economic sequelae.

In normal pregnancy, the onset of contractions is preceded by several weeks of cervical change characterised by decreased collagen and increased water content, identifiable clinically as effacement and shortening or cervical ‘ripening’. Cervical ripening is mediated by prostaglandin and cytokine secretion in the lower pole of the uterus and associated with an inflammatory cell infiltration. The later onset of uterine contractions is mediated by up regulation of a group of ‘contraction-associated proteins’ (CAPs) such as prostaglandin and oxytocin receptors, and gap junctions whose expression is repressed by progesterone. Preterm delivery prior to 32 weeks is associated with chorioamnionitis and ascending bacterial infection but recent studies have shown that most cases of early preterm labour cannot be attributed solely to ascending infection. The compound for use according to the present invention is intended to be used therapeutically to delay labour and/or inhibit uterine contractions in such subjects that are preterm. Said compound is also intended to optionally be used prophylactically to delay the on-set of PTL and/or preterm uterine contractions, as would be appreciated within the art. Thus in some embodiments, the compound for use according to the present invention may be used therapeutically and/or prophylactically in a subject at three or more weeks before the full gestation period of about 40 weeks, i.e. at 37 weeks of gestation or less.

In preferred compounds of Formula (I), each Z is independently selected from the group consisting of —CH₂—, —O—, and —C═C—;

In a preferred aspect of the invention, each X is independently C or N. Preferably, no more than 2 X in each ring is N.

In another preferred aspect of the invention, group A has the following formula:

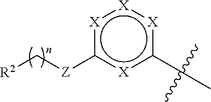

Therefore, particularly preferred examples of group A are:

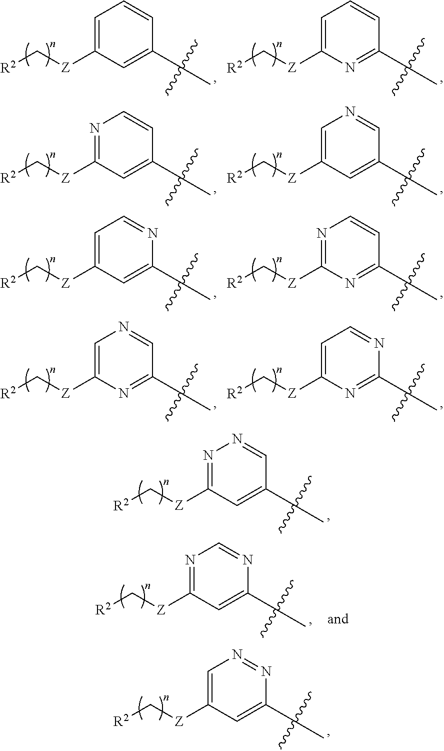

each of which are optionally substituted as described above.

In a particularly preferred aspect of the invention, A is:

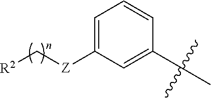

which is optionally substituted as described above.

In another preferred aspect of the invention, each X and Y is optionally independently substituted with a substituent independently selected from the group consisting of C₁-C₆ alkyl, C₁-C₆ alkoxy, (C₁-C₃ alkyl)-O—(C₁-C₃ alkyl), and halo, wherein the alkyl and alkoxy groups are optionally substituted with one or more substituents independently selected from the group consisting halo and —OH.

Particularly preferred compounds of Formula (I) are compounds of Formula (II):

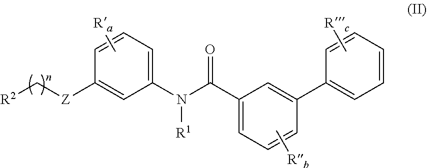

or a pharmaceutically acceptable salt, solvate, hydrate, tautomer, optical isomer, N-oxide, and/or prodrug thereof.

Each benzene ring in Formula (II) may be optionally substituted with one or more substituents represented by R′, R″, and R′″. The number of substituents on each ring is denoted by the integers a, b, and c.
a=0, 1, or 2, preferably 0 or 1, more preferably 0.
b=0, 1, or 2, preferably 1 or 2, more preferably 1.
c=0, 1, 2, or 3, preferably 1 or 2, more preferably 1.
R′, R″, and R′″ are each independently selected from the group consisting of C₁-C₆ alkyl, C₁-C₆ alkoxy, (C₁-C₃ alkyl)-O—(C₁-C₃ alkyl), and halo, wherein the alkyl and alkoxy groups are optionally substituted with one or more substituents independently selected from the group consisting halo and —OH.

More preferred compounds of Formula (I) are compounds of Formula (III):

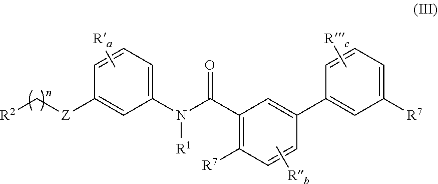

or a pharmaceutically acceptable salt, solvate, hydrate, tautomer, optical isomer, N-oxide, and/or prodrug thereof.

Each R⁷ is independently a halo atom, preferably F.
a=0, 1, or 2, preferably 0 or 1, more preferably 0; b=0, 1, or 2, preferably 0 or 1, more preferably 0; c=0, 1, 2, or 3, preferably 0 or 1, more preferably 0.
R′, R″, and R′″ are as defined above for Formula (II).

In preferred compounds of Formula (I), (II), and (Ill), R¹ is H.

It is also preferred that Y is —O—.

It is also preferred that n=1.

In also preferred that R² is —C(O)OH or —C(O)OR⁶, preferably —C(O)OH or —C(O)O— isopropyl.

Particular compounds for use according to the invention are:
2-(3-{3′,4-difluoro-[1,1′-biphenyl]-3-amido}phenoxy)acetic acid

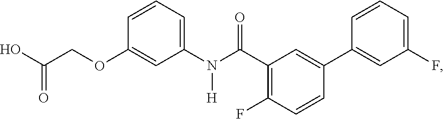 [SMILES: O=C(O)COc1cccc(NC(=O)c2cc(-c3cccc(F)c3)ccc2F)c1]

propan-2-yl 2-(3-{3′,4-difluoro-[1,1′-biphenyl]-3-amido}phenoxy)acetate

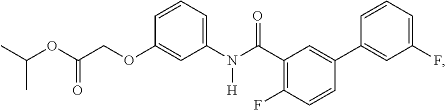 [SMILES: CC(C)OC(=O)COc1cccc(NC(=O)c2cc(-c3cccc(F)c3)ccc2F)c1]

or a pharmaceutically acceptable salt, solvate, hydrate, tautomer, N-oxide, and/or prodrug thereof.

The compounds for use according to the invention may include isotopically-labelled and/or isotopically-enriched forms of the compounds. The compounds for use according to the invention herein may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl.

The compounds for use according to the invention may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Throughout the present disclosure, a given chemical formula or name shall also encompass all pharmaceutically acceptable salts, solvates, hydrates, geometrical isomers, tautomers, optical isomers, N-oxides, and/or prodrug forms thereof. It is to be understood that the compounds for use according to the invention include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds for use according to the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g. having one or more stereocenters). All stereoisomers, such as enantiomers and diastereomers, are intended unless otherwise indicated. Compounds for use according to the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis- and trans-geometric isomers of the compounds for use according to the present invention are described and may be isolated as a mixture of isomers or as separated isomeric forms.

In the case of the compounds, which contain an asymmetric carbon atom, the invention relates to the D form, the L form, and D, L mixtures and also, where more than one asymmetric carbon atom is present, to the diastereomeric forms. Those compounds for use according to the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The term “prodrugs” refers to compounds that may be converted under physiological conditions or by solvolysis to a biologically active compound for use according to the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound for use according to the invention may be prepared by modifying functional groups, such as a hydroxy, amino or mercapto groups, present in a compound for use according to the invention in such a way that the modifications are cleaved, either in routine manipulation or in vivo, to the parent compound for use according to the invention. Examples of prodrugs include, but are not limited to, acetate, formate and succinate derivatives of hydroxy functional groups or phenyl carbamate derivatives of amino functional groups.

In some embodiments, the compound may be for use in the prevention of the onset of preterm-labour, in women at higher risk than normal.

The term “higher risk” refers to women who are more likely to experience PTL, and may include: women that have had a premature baby in the past; women that are pregnant with multiples, such as twins, triplets or more; and women that have physiological problems with their uterus or cervix at present and/or in the past. Having certain health conditions during pregnancy can also increase a subject's risk for PTL and premature birth, including: connective tissue disorders such as Ehlers-Danlos syndromes (EDS) and vascular Ehlers-Danlos syndrome (vEDS); high blood pressure and preeclampsia; diabetes; infections, including sexually transmitted infections (STIs), infections of the urinary tract, vagina and/or uterus; thrombophilias; and intrahepatic cholestasis of pregnancy (ICP). Other factors that may contribute to women being at higher risk than normal would be known within the art, but may further include: smoking; drinking alcohol; stress; drug abuse; domestic violence; low socioeconomic status; exposure to air pollution, radiation and chemicals; and being younger than 17 or older than 35.

The compound of the present invention is intended to be administered to a preterm pregnant subject in order to delay labour and/or inhibit uterine contractions. Thus in some embodiments, the compound of the present invention is for administration to a subject at less than or equal to 37 weeks gestation.

The term “administration” as used herein is intended to encompass all appropriate routes of administration to a subject known within the art. In a preferred embodiment, the compound is for administration to the subject via an intramuscular, intraperitoneal, subcutaneous or intravenous injection. In a most preferred embodiment, the compound is for administration via an oral or vaginal route.

In some further embodiments, the compound of the present invention may be co-administered simultaneously, sequentially or separately with other appropriate medicaments and/or therapeutic interventions. Such medicaments and/or therapeutic interventions may include one or more of: oxytocin receptor antagonists, prostaglandin receptor antagonists, beta-adrenergic receptor agonists, nitrogen oxide donors, magnesium sulphate, prostaglandin-synthase inhibitors, non-steroidal anti-inflammatory drugs (NSAIDs), small molecule and other anti-inflammatory drugs, calcium channel blockers, progesterone, 17-a-hydroxyprogesterone caproate, and progesterone analogues.

In a second aspect of the invention, there is provided a selective prostaglandin E2 receptor (EP2) agonist for use in delaying labour and/or inhibiting uterine contractions in a preterm pregnant subject.

It is intended to be appreciated by the skilled person that such a selective EP2 agonist may be a compound as defined by the first aspect of the invention.

The term “selective EP2 agonist” as used herein relates to agonists that selectively activate the anti-labour cyclic AMP (cAMP) pathway (or Gαs pathway) of EP2. EP2 is a dually coupled G-protein coupled receptor (GPCR) capable of activating downstream signalling pathways via Gαs (anti-labour) and Gαq/11 (pro-labour). Activation of the EP2 receptor via agonists increases the Gαs cell signalling pathway messenger cAMP, which relaxes smooth muscle cells, including smooth muscle in the uterus or myometrium. In the full-term pregnant uterus, wherein full-term refers to a pregnant subject at 37 weeks or more gestation, it is known that using the non-selective or “biased” agonists such as butaprost can activate EP2. However, the inventors have demonstrated that, surprisingly, EP2, in addition to activating well established Gαs anti-labour pathways (by increasing the second messenger signalling molecule cAMP to relax the smooth muscle of the pregnant uterus), also activates opposing Gαq/11 pro-labour/pro-inflammatory pathways that involve intracellular calcium release, via a distinct G-protein signalling pathway. Once a patient who is at term pregnancy goes into labour, the anti-labour cAMP pathway activated by EP2 decreases while the Ca²⁺-related pro-labour pathway is maintained (Kandola et al. 2014, Endocrinology 155: 2 (605-617). The inventors also demonstrate that these dual and opposing pathways activated by EP2 are present even in the pregnant pre-term uterus, at fewer than 37 weeks gestation.

As would be appreciated by the skilled person, a selective EP2 agonist that only activates the anti-labour cAMP pathway of EP2 would thus serve as a safe, efficacious and cost-effective treatment for PTL. Such a selective EP2 agonist would not induce signals that increase inflammation, as observed with other EP2 agonists within the prior art.

Thus in some embodiments, the selective EP2 agonist selectively activates the anti-labour cAMP pathway of EP2, without inducing inflammatory signals.

Further surprising findings by the inventors show an ability of EP2 to maintain its pro-labour/pro-inflammatory pathway via crosstalk and/or interaction with the oxytocin receptor, which thus far has been the primary focus of pre-term labour targets. It would thus be appreciated that a selective EP2 agonist which further inhibits oxytocin-mediated increases in inflammation via crosstalk and/or interactions between both EP2 and an oxytocin receptor would function as an effective compound for the inhibition of contractility through selective activation of the anti-labour cAMP EP2 pathway, as well as further inhibition of the pro-inflammatory environment of PTL. Such a compound has not been known in the prior art, and would demonstrate a current therapeutic need in PTL that could protect the developing brain and lungs of a baby.

Thus, in some embodiments, the selective EP2 agonist for use according to the present invention further inhibits oxytocin-mediated increases in inflammation. In a preferred embodiment, the agonist further inhibits oxytocin-mediated increases in inflammation via cross-talk between EP2 and an oxytocin receptor.

It is envisaged that the selective EP2 agonist may be long-lasting in its action, further suggesting that a single treatment would only be needed for the prevention of the onset of PTL and/or delaying of labour and/or inhibition of uterine contractions.

In some embodiments, the selective EP2 agonist may be for use in the prevention of the onset of preterm-labour, in women at higher risk than normal.

It is envisaged that the selective EP2 agonist for use according to the present invention may be PGN-9856. PGN-9856 is a long-acting EP2 agonist, known within the art for its use of treatment in the context of the eye and, specifically, glaucoma. PGN-9856 was discovered by the inventors to be a long-acting EP2 agonist that, surprisingly, only activates the anti-labour cAMP pathway, without inducing signals that increase inflammation as observed with other EP2 agonists. In addition, the inventors discovered crosstalk between EP2 and oxytocin receptors, which led to the demonstration that PGN-9856 inhibits oxytocin-mediated increases in inflammation.

Thus in a preferred embodiment, the selective EP2 agonist for use according to the present invention is PGN-9856, as shown below:

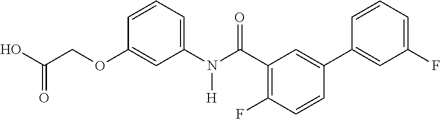 [SMILES: O=C(O)COc1cccc(NC(=O)c2cc(-c3cccc(F)c3)ccc2F)c1]

In a most preferred embodiment, the selective EP2 agonist for use according to the present invention is the isopropyl form of the above compound, designated PGN-9856i, as shown below:

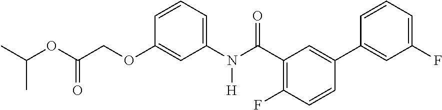 [SMILES: CC(C)OC(=O)COc1cccc(NC(=O)c2cc(-c3cccc(F)c3)ccc2F)c1]

In a third aspect of the invention, there is provided a pharmaceutical formulation for use in delaying labour and/or inhibiting uterine contractions in a preterm pregnant subject, wherein the pharmaceutical composition comprises a compound of Formula (I) as defined in the first aspect and one or more pharmaceutically acceptable carriers or excipients.

For clinical use, the compounds disclosed herein are formulated into pharmaceutical compositions (or formulations) for various modes of administration. It will be appreciated that compounds of the invention may be administered together with a physiologically acceptable carrier, excipient, and/or diluent (i.e. one, two, or all three of these). The pharmaceutical compositions disclosed herein may be administered by any suitable route, preferably by via an intramuscular, intraperitoneal, subcutaneous or intravenous injection, or via an oral or vaginal route, including a pessary or gel. Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories, pessaries, gels or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

In a fourth aspect, there is provided a method for delaying labour and/or inhibiting uterine contractions in a preterm pregnant subject, the method comprising the step of administering to a patient in need thereof a therapeutically effective amount of a compound of Formula (I) as defined in the first aspect, an agonist as defined in the second aspect, or a pharmaceutical composition as defined in the third aspect of the invention.

“A therapeutically effective amount” refers to an amount of a compound for use according to the invention that confers a therapeutic effect on the treated subject. The therapeutic effect may be objective (i.e. measurable by some test or marker) or subjective (i.e. subject gives an indication of or feels an effect).

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. It is preferred that the subject is human.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

The invention provides a method of monitoring treatment progress. The method includes the step of determining a level of diagnostic marker (Marker) (e.g. any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

A level of Marker or Marker activity in a subject may be determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, is ELISA, radiolabeling/assay techniques, blotting/chemiluminescence methods, real-time PCR, and the like.

In a fifth aspect, there is provided a use of a compound of Formula (I) as defined in the first aspect, an agonist as defined in the second aspect, or a pharmaceutical composition as defined in the third aspect, in the manufacture of a medicament for delaying labour and/or inhibiting uterine contractions in a preterm pregnant subject.

### Definitions

“Optional” or “optionally” means that the subsequently described event or circumstance may, but need not, occur, and that the description includes instances where the event or circumstance occurs and instances in which it does not.

The term “C₁-C₆ alkyl” denotes a straight, branched or cyclic or partially cyclic alkyl group having from 1 to 6 carbon atoms, i.e. 1, 2, 3, 4, 5 or 6 carbon atoms. For the “C₁-C₆ alkyl” group to comprise a cyclic portion it should be formed of 3 to 6 carbon atoms. For parts of the range “C₁-C₆ alkyl” all subgroups thereof are contemplated, such as C₁-C₅ alkyl, C₁-C₄ alkyl, C₁-C₃ alkyl, C₁-C₂ alkyl, C₁ alkyl, C₂-C₆ alkyl, C₂-C₅ alkyl, C₂-C₄ alkyl, C₂-C₃ alkyl, C₂ alkyl, C₃-C₆ alkyl, C₃-C₅ alkyl, C₃-C₄ alkyl, C₃ alkyl, C₄-C₆ alkyl, C₄-C₅ alkyl, C₄ alkyl, C₅-C₆ alkyl, C₅ alkyl, and C₆ alkyl. Examples of “C₁-C₆ alkyl” include methyl, ethyl, n-propyl, isopropyl, cyclopropyl, n-butyl, isobutyl, sec-butyl, t-butyl, cyclobutyl, cyclopropylmethyl, and straight, branched or cyclic or partially cyclic pentyl and hexyl etc.

When a term denotes a range, for instance “1 to 6 carbon atoms” in the definition of C₁-C₆ alkyl, each integer is considered to be disclosed, i.e. 1, 2, 3, 4, 5 and 6.

The term “C₂-C₆ alkenyl” denotes a straight, branched or cyclic or partially cyclic alkyl group having at least one carbon-carbon double bond, and having from 2 to 6 carbon atoms. The alkenyl group may comprise a ring formed of 3 to 6 carbon atoms. For parts of the range “C₂-C₆ alkenyl” all subgroups thereof are contemplated, such as C₂-C₅ alkenyl, C₂-C₄ alkenyl, C₂-C₃ alkenyl, C₂ alkenyl, C₃-C₆ alkenyl, C₃-C₅ alkenyl, C₃-C₄ alkenyl, C₃ alkenyl, C₄-C₆ alkenyl, C₄-C₆ alkenyl, C₄ alkenyl, C₅-C₆ alkenyl, C₅ alkenyl, and C₆ alkenyl. Examples of “C₂-C₆ alkenyl” include 2-propenyl, 2-butenyl, 3-butenyl, 2-methyl-2-propenyl, 2-hexenyl, 5-hexenyl, 2,3-dimethyl-2-butenyl.

The term “C₂-C₆ alkynyl” denotes a straight, branched or cyclic or partially cyclic alkyl group having at least one carbon-carbon triple bond, and having from 2 to 6 carbon atoms. The alkynyl group may comprise a ring formed of 3 to 6 carbon atoms. For parts of the range “C₂-C₆ alkynyl” all subgroups thereof are contemplated, such as C₂-C₅ alkynyl, C₂-C₄ alkynyl, C₂-C₃ alkynyl, C₂ alkynyl, C₃-C₆alkynyl, C₃-C₅ alkynyl, C₃-C₄ alkynyl, C₃ alkynyl, C₄-C₆ alkynyl, C₄-C₅ alkynyl, C₄ alkynyl, C₅-C₆ alkynyl, C₅ alkynyl, and C₆ alkynyl. Examples of “C₂-C₆ alkynyl” include 2-propynyl, 2-butynyl, 3-butynyl, 2-pentynyl, 3-methyl-4-pentynyl, 2-hexynyl, 5-hexynyl etc.

The term “C₁-C₆ alkoxy” denotes —O—(C₁-C₆alkyl) in which a C₁-C₆ alkyl group is as defined above and is attached to the remainder of the compound through an oxygen atom. Examples of “C₁-C₆ alkoxy” include methoxy, ethoxy, n-propoxy, isopropoxy, n-butoxy, isobutoxy, sec-butoxy, t-butoxy and straight- and branched-chain pentoxy and hexoxy.

The term “5-membered heteroaryl” denotes an aromatic monocyclic heteroaromatic ring system having 5 ring atoms in which 1 to 4 of the ring atoms are carbon and one or more of the ring atoms are selected from nitrogen, sulphur, and oxygen. Examples of “5-membered heteroaryl” include furyl, pyrrolyl, thienyl, oxazolyl, isoxazolyl, imidazolyl, thiazolyl, isothiazolyl, and tetrazolyl.

The term “halo atom” means a halogen atom, and is preferably F, Cl, Br or I.

Compounds for use according to the invention may be disclosed by the name or chemical structure. If a discrepancy exists between the name of a compound and its associated chemical structure, then the chemical structure prevails.

### Example 1

EP2 is a dually coupled GPCR capable of activating downstream signalling pathways via Gαs (anti-labour inducing) and Gαq/11 (pro-labour inducing). In order to demonstrate the activation of the anti-labour pathway of EP2, cAMP was measured as a readout of Gαs activation in HEK 293 cells stably expressing human FLAG-tagged EP2.

HEK 293 cells were maintained in DMEM (Dulbecco's Modified Eagle Medium) containing 10% fetal bovine serum and 100 μ/ml penicillin-streptomycin, and cultured in 75 cm flasks at 37° C. in 95% air and 5% CO₂. Cells at 95% confluency were passaged using 0.25% trypsin with 0.02% EDTA in phosphate-buffered saline (PBS).

All statistical data analyses were performed in GraphPad Prism 8. An unpaired t-test was used for comparison of two groups. In cases where patient data had been expressed as fold change over basal or agonist response, a one-sample t-test was performed to allow comparison against a bounded value. For comparison of multiple groups a one-way ANOVA was used followed by either Tukey's, Dunnet's or Sidak's multiple comparison post-hoc tests. For each test, P<0.05 was considered significant.

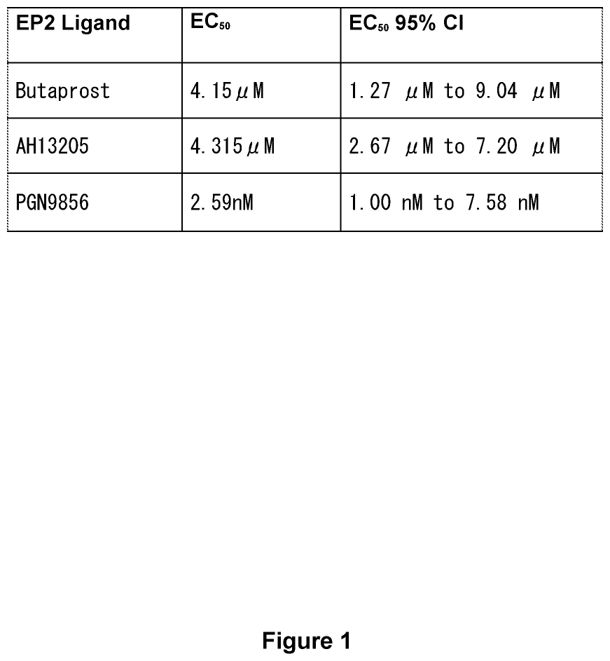

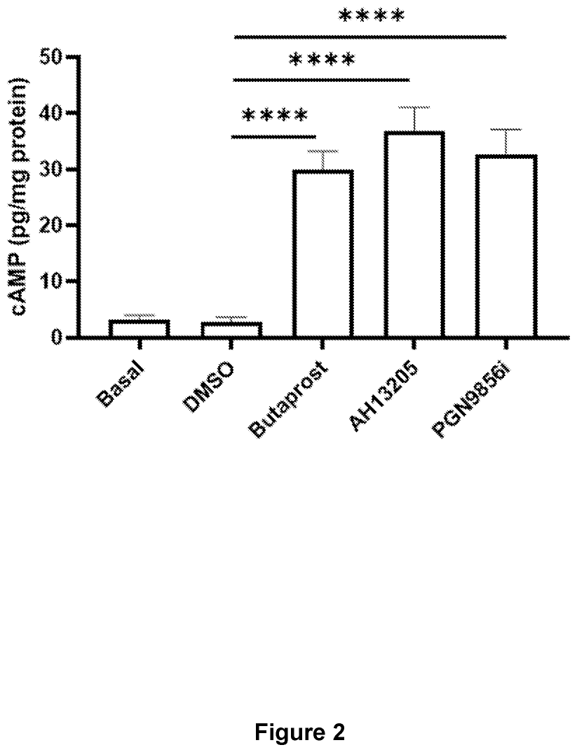

Dose-response curves were analysed and the EC50 was calculated for three highly selective EP2 agonists: butaprost, AH13205 and PGN9856i. PGN9856i exhibited the greatest potency, by three orders of magnitude (FIG. 1). The maximal dose for each ligand was found to be equally efficacious, and there was no observed significant difference between ligands after 5 minutes of stimulation (FIG. 2).

### Example 2

To measure Gαq/11 activation, HEK 293 cells stably expressing human FLAG-EP2 were pre-labelled with a Ca2+ indicator dye and acutely stimulated with each ligand.

HEK 293 cells, prepared following the same methodology as outlined in Example 1, were seeded onto Mattek dishes with 14 mm by 1.5 mm glass coverslips. Cells were incubated with Fluo-4am Ca2+ indicator (ThermoFisher) as per manufacturer's instructions. (1-hour, 100 nM). Time-series images were acquired using a Leica SP5 confocal microscope, Leica LAS AF image acquisition software and a 488 nm excitation laser. HEK cells were imaged using a 10× dry objective. EP2 agonists (butaprost 10 μM, AH13205 10 μM or PGN9856i 100 nM) were added following measurement of basal fluorescence. Raw files were analysed using Fiji Time series analyser plugin to find the maximal fluorescent intensity, which was then averaged across cells in each condition.

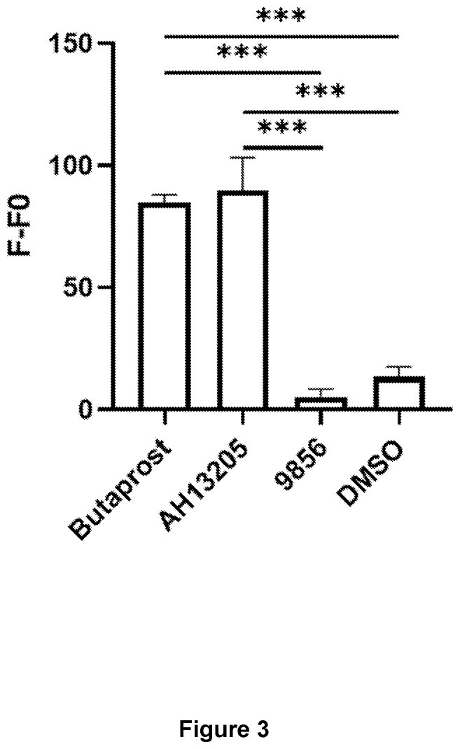

The maximal fluorescence intensity was measured by live confocal microscopy. PGN9856i (“9856”) was the only ligand unable to elicit intracellular Ca2+ release, while both AH13205 and butaprost enhanced Ca2+ significantly above vehicle indicating PGN9856i to be unable to activate Gαq/11 (FIG. 3).

### Example 3

COX-2 is an inflammatory mediator that drives PGE2 production. The inventors had previously shown EP2-dependent COX-2 release to be downstream of Ca2+ release in primary human myometrium and that 6-hour agonist treatment is necessary for increasing COX-2 levels and PGE2 secretion (Kandola et al, 2014). HEK 293 cells stably expressing human FLAG-EP2 were stimulated for 6 hours with an EP2 agonist (butaprost 10 μM, AH13205 10 μM or PGN9856i 100 nM) or DMSO. 1 mL media was collected from COX-2 experiments and immediately frozen at −80 degrees Celsius. Samples were defrosted at room temperature and vortexed before quantification of IL-6 concentrations via ELISA as per manufacturer's instructions (Enzo Lifesciences).

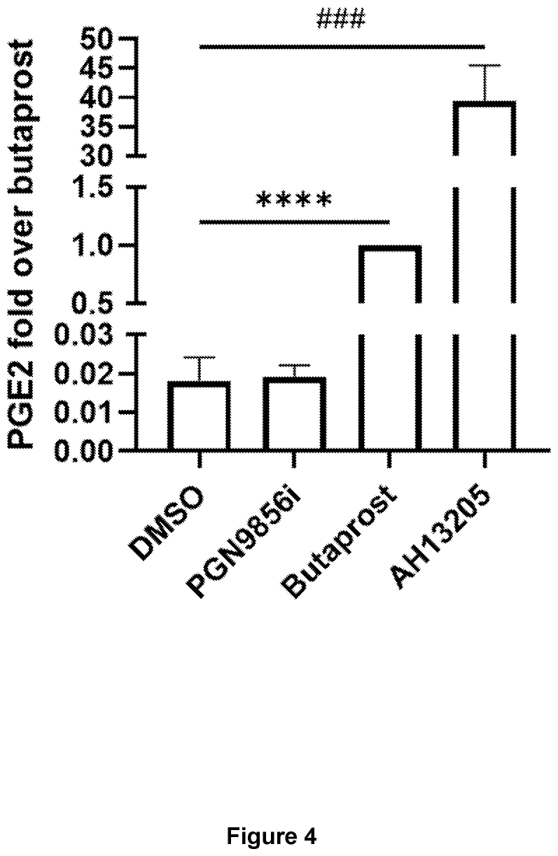

While butaprost and AH13205 were shown to be efficacious activators of the pathway, PGN9856i was unable to increase PGE2 levels over basal levels (FIG. 4).

### Example 4

In the myometrium EP2 is an important regulator of parturition. To assess if PGN9856i, butaprost and AH13205 induce similar second messenger profiles in the myometrium as HEK 293 cells, primary human myocytes cultured from term pregnant, non-labouring, myometrium, were treated with each ligand and intracellular cAMP, calcium and PGE2 release was measured.

Myometrial tissue was acquired at St. Mary's hospital, Queen Charlotte and Chelsea hospital and Charing Cross hospital, London. Tissue was obtained from women provided with informed written consent with the approval from the Riverside Research Ethics Committee (RREC 3357, 1997-5089, 19/LO/1657) and experiments were carried out under the committee's guidelines and recommendations. Tissues were obtained from term (38+0-40 weeks gestation) pregnant women undergoing elective caesarean from the upper margin of the incision made at the lower segment of the uterus, before or after the onset of labor. Tissues taken from women in early labour (<3 cm cervical dilation) or late labour (>3 cm cervical dilation) with/without induction with syntocinon, an oxytocin analogue. Tissue was only taken from uncomplicated, singleton pregnancies.

Tissue was stored at 4° C. in phosphate buffered saline until fine dissection with scalpels and dissociation at 37° C. for 1 hour in a sterile-filtered mix of Dulbecco's modified Eagle's medium (DMEM) (Sigma Aldrich)/Ham's F-12 Nutrient Mixture (Sigma Aldrich) and serum-free DMEM (Sigma Aldrich) (1:1 v/v) containing collagenase 1A (Sigma Aldrich)(1 mg/ml), collagenase X (Sigma Aldrich)(1 mg/ml) and bovine serum albumin (Sigma Aldrich) (2 mg/ml). Dissociation was ended using DMEM 10% fetal bovine serum (Sigma Aldrich) and cell suspension was obtained using a 40-μm cell strainer before centrifugation at 3000 rpm for 5 minutes. Each cell pellet was re-suspended in DMEM containing 10% fetal bovine serum and 100 U/ml penicillin-streptomycin (Sigma Aldrich) and cultured in 75 cm flasks at 37° C. in 95% air and 5% CO₂. Cells at 95% confluency were passaged using 0.25% trypsin with 0.02% EDTA in phosphate-buffered saline. Cells taken from women in labour were used at passage 0, those from non-labouring women were used until passage 5.

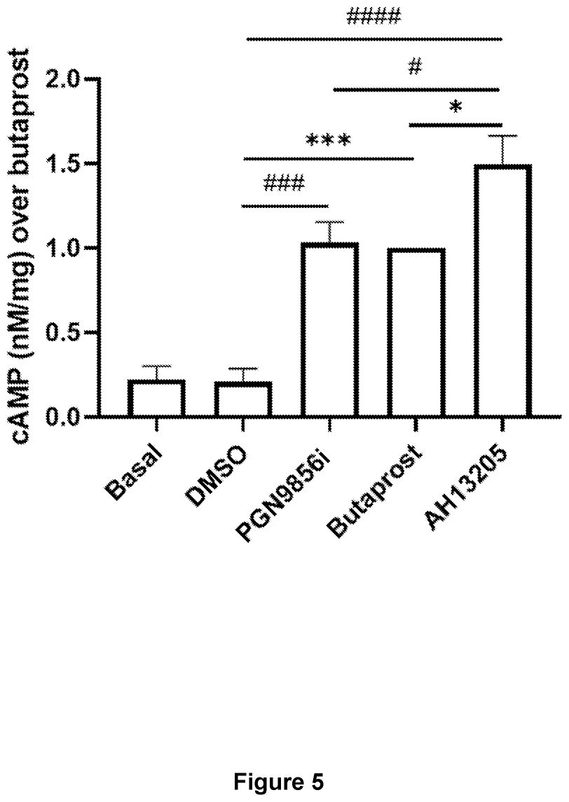

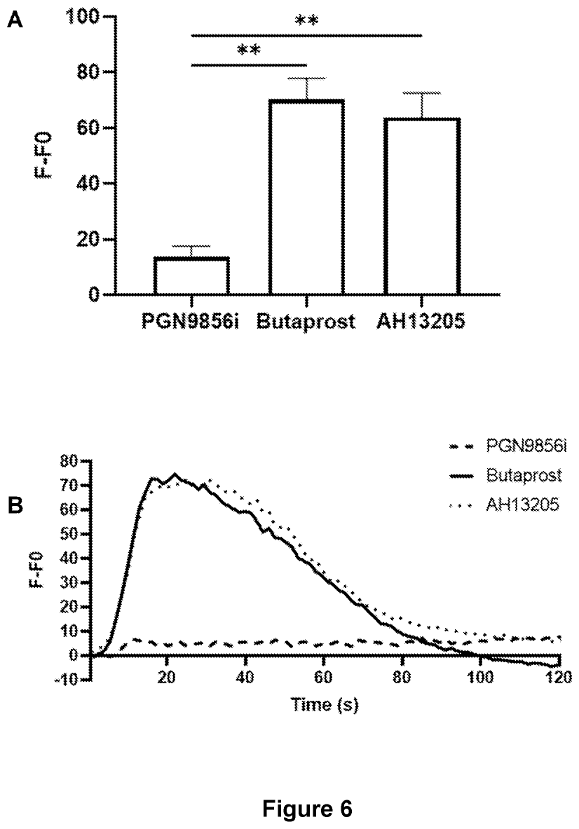

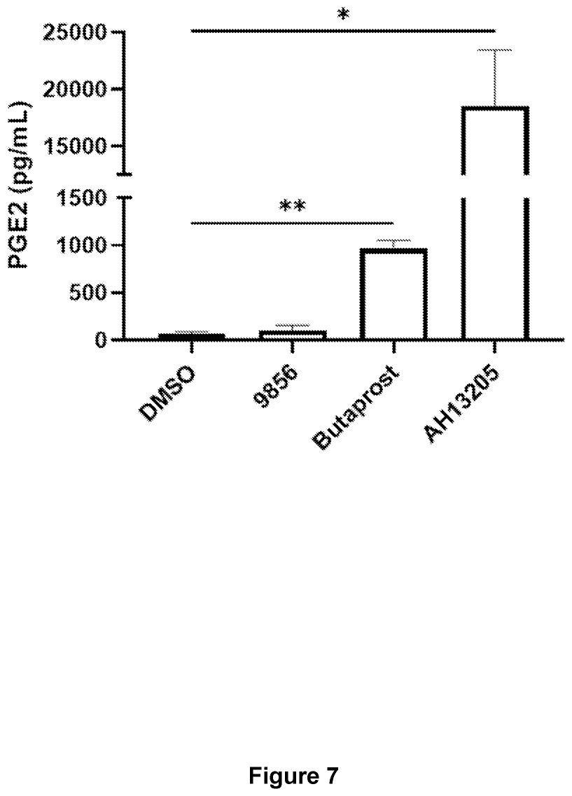

The ligands were all able to elicit release of intracellular cAMP (FIG. 5). However, PGN9856i, unlike butaprost and AH13205, was unable to activate calcium (FIG. 6) or PGE2 (FIG. 7) pathways, suggesting that in myometrial cells, in addition to HEK 293, PGN9856i is unable to activate Gαq/11 pathways.

### Example 5

Oxytocin (OT) and its receptor (OTR) is a key driver of human labour and a target in tocolysis to manage pre-term labour. EP2 signalling is reprogrammed during labour to maintain its pro-inflammatory pathway via unknown mechanisms (Kandola et al, 2014). To identify underlying mechanisms, myometrial samples were taken from women being delivered by Caesarean section, during early (cervical dilation <3 cm) or late labour (cervical dilation >3 cm). Samples were also obtained with or without labour induction with intravenous OT. The primary myocyte cultures were stimulated with either butaprost, or isoproterenol, a non-selective Gαs-coupled β-adrenergic receptor (PAR) agonist.

Myometrial cells taken from women in different labouring groups were treated for 6-hours with butaprost. Confluent myometrial cells were lysed in RIPA buffer containing 1% Triton x-100, 1% Sodium deoxycholate, 0.1% SDS, 150 mM NaCl, 10 mM Tris, 1 mM EDTA plus 1 mM sodium orthovanadate and one Pierce EDTA-Free protease inhibitor mini tablet. 20-40 μg of lysate as determined via Bradford assay plus Laemmli buffer (0.5M pH 6.8 Tris, 10% SDS, 1% Bromophenol blue, 2% B-ME, 20% Glycerol) were separated with 1×SDS running buffer at 140V on SDS-PAGE gels polymerized with TEMED and ammonium persulfate: separating gel (40% poly, 1M pH 8.8 Tris, 10% SDS), resolving gel (40% poly, 1M Tris (pH 6.8), 10% SDS). Proteins were transferred onto nitrocellulose membranes before blocking in 5% Milk TBS-T for 30 minutes and incubated overnight at 4° C. with primary antibody and blocking buffer (1:1000). Membranes were then incubated in TBS-T with appropriate secondary antibody for 1-3 hours (1:2000) before signal detection using HRP substrate and chemiluminescent imager (ImageQuant LAS 4000).

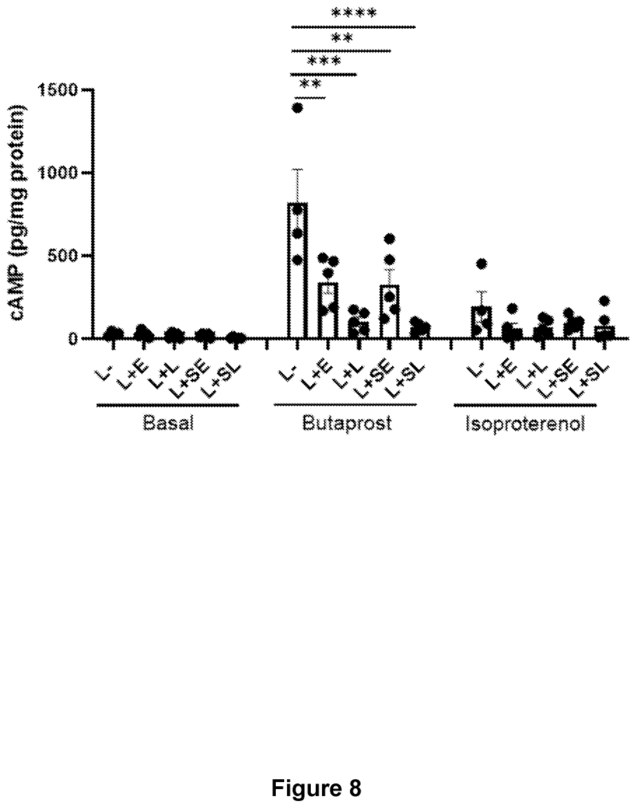

Butaprost, but not isoproterenol-induced cAMP signalling, was significantly attenuated in all labouring groups, with butaprost-stimulated cultures exhibited a 2.5-fold and 10-fold reduction between non-labouring and spontaneous early and late labour samples respectively (FIG. 8). Butaprost-induced increases in COX-2 were maintained across non-labouring and labouring groups and exhibited a significant increase in all early stage labouring groups, which was not apparent in either late stage labouring group (FIG. 8A-B).

Together, these results indicate that EP2 activity is not only changed to favour a pro-inflammatory pathway over the anti-labour cAMP pathway at each stage of labour, but that there is specific targeted reprogramming of EP2 over certain other Gαs-coupled GPCRs. This reprogramming of butaprost-induced signalling by OTR, and enhancement of pro-inflammatory pathways, is due to EP2 crosstalk with the Gαi-coupled OTR.

### Example 6

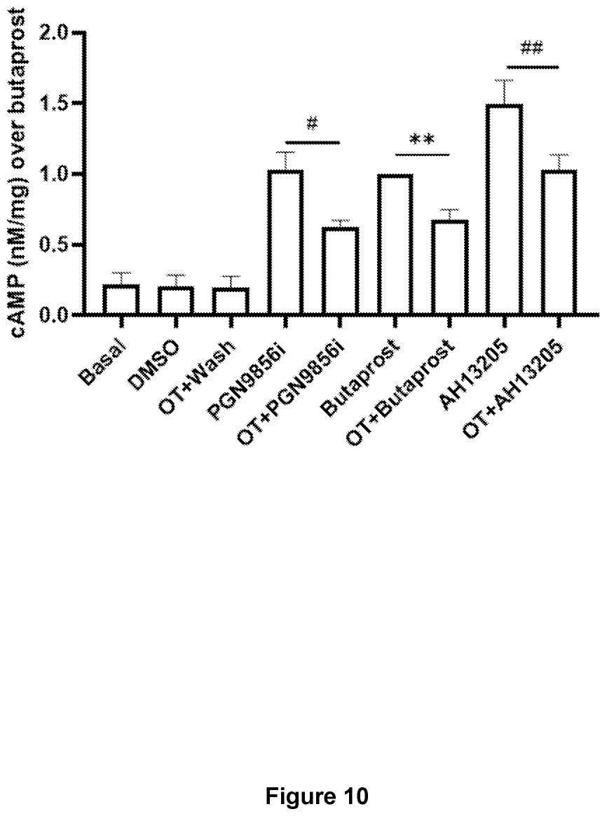

To directly demonstrate these changes were due to OT, non-labouring myometrial cells were pre-treated with/without 1-hour OT before washing with PBS and subsequent stimulation with either PGN9856i, AH13205 or butaprost. OTR activation significantly reduced all three EP2 ligands' ability to increase intracellular cAMP concentrations (FIG. 10).

Confluent myometrial cells were treated with/without OT (100 nM) for 1 hour before 3 PBS washes and the addition of 0.5 μM IBMX (3-isobutyl-1-methylxanthine) in PBS for 5 minutes at 37° C. This was exchanged for 0.5 μM IBMX in PBS plus either DMSO, butaprost (10 μM), AH13205 (10 μM) or PGN9856i (100 nM) for an additional 5-minutes in HEK cells. Cells were lysed in Cisbio cAMP lysis buffer with 0.2% TritonX-100 and centrifuged for 15 minutes at 16,000 g. Lysates were normalized to protein concentration determined by Coomassie (Bradford) Protein Assay Kit and analysed as per manufacturers' protocol (Cisbio Gαs dynamic assay) using a PHERAstar FSX.

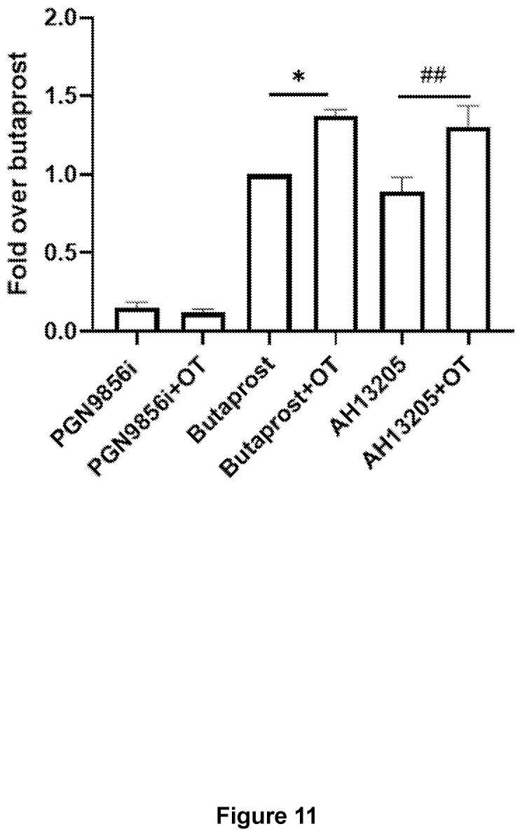

Intracellular calcium release was measured with/without OT pre-treatment which revealed OT to significantly enhance the calcium response following butaprost and AH13205 stimulation (FIG. 11). PGN9856i, however, was unable to elicit a response, a property that was not altered with OTR activation.

PGE2 is an inflammatory mediator upstream of COX-2, and upregulation of its release is significantly associated with labour. Myometrial cells were treated for 6-hours with EP2 agonist (butaprost 10 μM, AH13205 10 μM or PGN9856i 100 nM) or DMSO with/without 1-hour OT (100 nM) pre-treatment. 1 mL media was collected from COX-2 experiments and immediately frozen at −80 degrees Celsius. Samples were defrosted at room temperature and vortexed before quantification of IL-6 concentrations via ELISA as per manufacturer's instructions (Enzo Lifesciences).

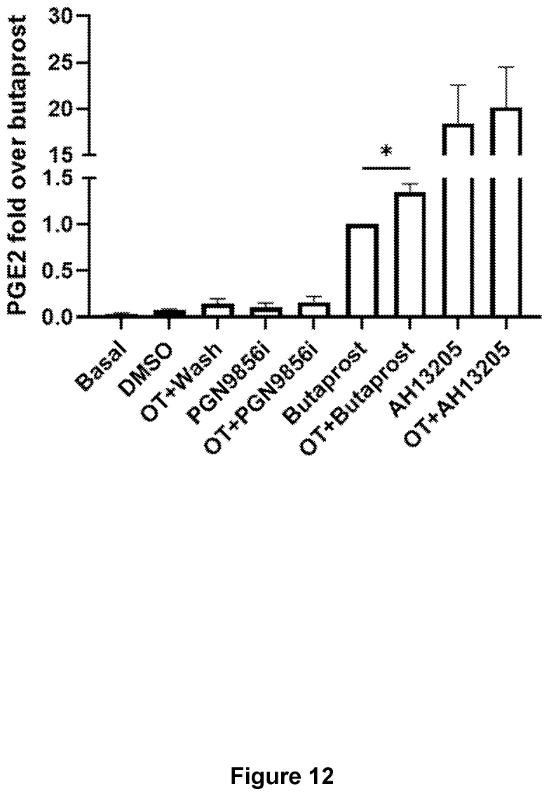

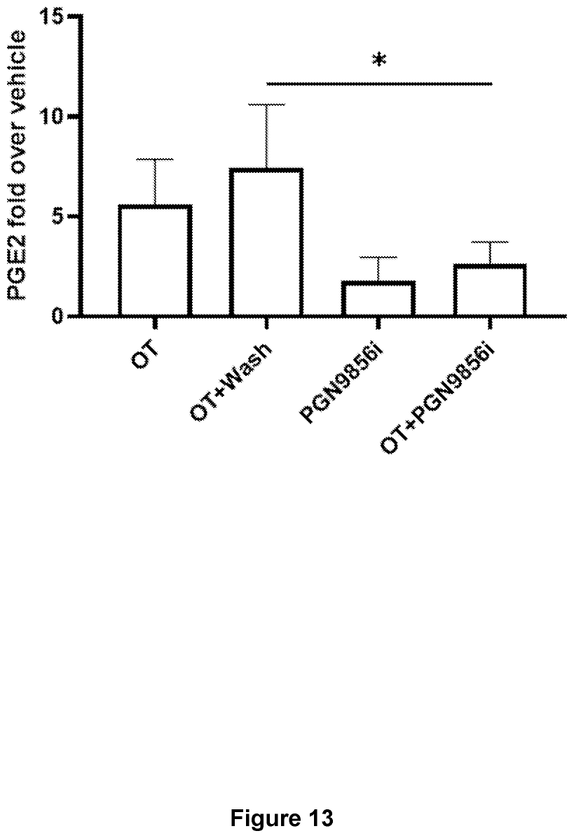

While butaprost significantly enhanced PGE2 release in myometrial cells, a response which was significantly increased by OT, PGN9856i was unable to cause PGE2 release, a property that was unchanged by OT pre-treatment (FIG. 12). When myometrial cells were treated with OT followed by 6-hour PGN9856i, PGN9856i was able to significantly reduce OT-mediated PGE2 release (FIG. 13).

Together, this data demonstrates that OTR activation specifically reduces the ability of EP2 to decrease cAMP, an anti-labour mediator, and increase pro-inflammatory, pro-labour pathways, when activated by butaprost or AH13205. However, when activated by PGN9856i, although cAMP release is reduced with OT treatment, PGN9856i is unique in that it maintains anti-inflammatory responses even after activation of OTR, and even inhibits pro-inflammatory responses of OTR.

1. A method of prophylactically delaying the on-set of labour and/or uterine contractions in a preterm pregnant subject, the method comprising administering to the subject a compound of Formula (I): 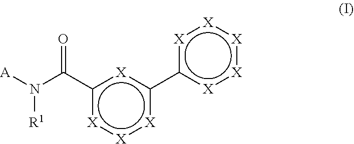
or a pharmaceutically acceptable salt, solvate, hydrate, tautomer, optical isomer, N-oxide, and/or prodrug thereof,
wherein in Formula (I):
A is: 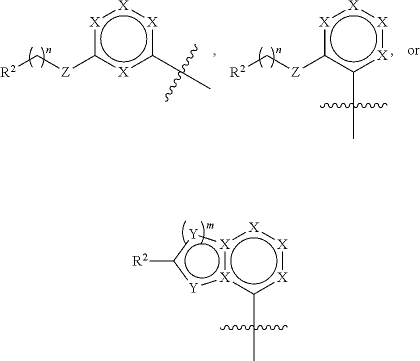
m is 1 or 2;
each X and Y is independently selected from C, N, O, and S, wherein each X and Y is optionally independently substituted with a substituent selected from the group consisting of:
(i) C₁-C₆ alkyl, C₂-C₆ alkenyl, C₂-C₆ alkynyl, C₁-C₆ alkoxy, and (C₁-C₃ alkyl)-O—(C₁-C₃ alkyl), each of which is optionally substituted with one or more substituents independently selected from halo, —OH, —NR³R⁴, and oxo; and
(ii) halo, —OH, —CN, —NO₂, —SO₂, —NR³R⁴, —C(O)NR₃R⁴—C(O)OR⁵, and —C(O)R⁵;
each Z is independently selected from the group consisting of —CH₂—, —O—, —S—, —NH—, and —C═C—, preferably —CH₂—, —O—, and —C═C—;
each n is independently 0, 1, 2, or 3;
R¹ is H or is selected from the group consisting of C₁-C₆ alkyl, C₂-C₆ alkenyl, C₂-C₆ alkynyl, each of which is optionally substituted with one or more substituents independently selected from halo, —NR³R⁴, —OH, and oxo;
each R² is independently selected from the group consisting of —C(O)OH, —C(O)OR⁶, —OC(O)R⁶, —C(O)R⁶, —C(O)NHR⁶, —NHC(O)R⁶, —OR⁶, —R⁶, and 5-membered heteroaryl, wherein the 5-membered heteroaryl is optionally substituted with one or more substituents independently selected from C₁-C₆ alkyl and halo, the C₁-C₆ alkyl being optionally substituted with one or more halo atoms;
R³, R⁴, and R⁵ are each independently selected from the group consisting of H, and C₁-C₆ alkyl, wherein the C₁-C₆ alkyl is optionally substituted with one or more halo atoms;
each R⁶ is independently selected from the group consisting of H and C₁-C₆ alkyl, wherein the C₁-C₉ alkyl is optionally substituted with one or more substituents independently selected from halo and —OH.

2. The method according to claim 1, wherein each X is independently C or N, preferably wherein no more than 2 X in each ring is N.

3. The method according to claim 1, wherein each X and Y is optionally independently substituted a substituent independently selected from the group consisting of C₁-C₆ alkyl, C₁-C₆ alkoxy, (C₁-C₃ alkyl)-O—(C₁-C₃ alkyl), and halo, wherein the alkyl and alkoxy groups are optionally substituted with one or more substituents independently selected from the group consisting halo and —OH.

4. The method according claim 1, wherein the compound of Formula (I) is a compound of Formula (II): 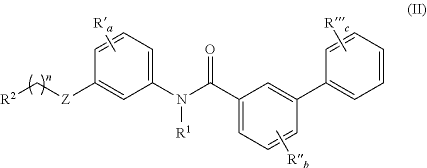
or a pharmaceutically acceptable salt, solvate, hydrate, tautomer, optical isomer, N-oxide, and/or prodrug thereof, wherein
a=0, 1, or 2, preferably 0 or 1, more preferably 0;
b=0, 1, or 2, preferably 1 or 2, more preferably 1;
c=0, 1, 2, or 3, preferably 1 or 2, more preferably 1;
R′, R″, and R′″ are each independently selected from the group consisting of C₁-C₆ alkyl, C₁-C₆ alkoxy, (C₁-C₃ alkyl)-O—(C₁-C₃ alkyl), and halo, wherein the alkyl and alkoxy groups are optionally substituted with one or more substituents independently selected from the group consisting halo and —OH.

5. The method according to claim 4, wherein the compound of Formula (I) is a compound of Formula (III): 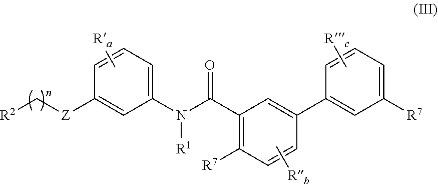
or a pharmaceutically acceptable salt, solvate, hydrate, tautomer, optical isomer, N-oxide, and/or prodrug thereof,
wherein each R⁷ is halo, preferably F;
a=0, 1, or 2, preferably 0 or 1, more preferably 0;
b=0, 1, or 2, preferably 0 or 1, more preferably 0;
c=0, 1, 2, or 3, preferably 0 or 1, more preferably 0.

6. The method according to claim 1, wherein R¹ is H.

7. The method according to claim 1, wherein Y is —O—.

8. The method according to claim 1, wherein n=1.

9. The method according to claim 1, wherein R² is —C(O)OH or —C(O)OR⁶, preferably —C(O)OH or —C(O)O-isopropyl.

10. The method according to claim 1, wherein the compound of Formula (I) is 2-(3-{3′,4-difluoro-[1,1′-biphenyl]-3-amido}phenoxy)acetic acid 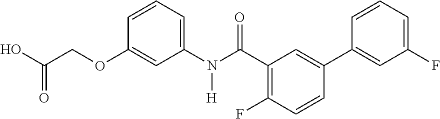 [SMILES: O=C(O)COc1cccc(NC(=O)c2cc(-c3cccc(F)c3)ccc2F)c1]
or a pharmaceutically acceptable salt, solvate, hydrate, tautomer, N-oxide, and/or prodrug thereof.

11. The method according to claim 1, wherein the compound of Formula (I) is propan-2-yl 2-(3-{3′,4-difluoro-[1,1′-biphenyl]-3-amido}phenoxy)acetate 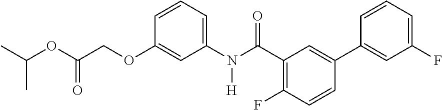 [SMILES: CC(C)OC(=O)COc1cccc(NC(=O)c2cc(-c3cccc(F)c3)ccc2F)c1]
or a pharmaceutically acceptable salt, solvate, hydrate, tautomer, N-oxide, and/or prodrug thereof.

12. The method according to claim 1, wherein the preterm pregnant subject is a woman at higher risk than normal for onset of preterm-labour.

13. The method according to claim 1, wherein the compound of Formula (I) is administered to the subject at less than or equal to 37 weeks gestation.

14. The method according to claim 1, wherein the compound of Formula (I) is administered to the subject via an intramuscular, intraperitoneal, subcutaneous or intravenous injection.

15-22. (canceled)

23. A pharmaceutical formulation for use prophylactically to delay the on-set of labour and/or uterine contractions in a preterm pregnant subject, wherein the pharmaceutical composition comprises a compound of Formula (I) as defined in claim 1, and one or more pharmaceutically acceptable carriers or excipients.

24-25. (canceled)
